(19) United States
(12) Plant Patent Application Publication
(10) Pub. No.: US 2025/0031590 P1
(43) Pub. Date: Jan. 23, 2025

(54) Mandarin Tree Named 'AC41114'

(50) ;

(71) Applicant: C&B Pressler Family Trust, Bundaberg (AU)

(72) Inventor: Craig Robert Pressler, Bundaberg (AU)

(73) Assignee: C&B Pressler Family Trust, Bundaberg (AU)

(21) Appl. No.: 18/771,728

(22) Filed: Jul. 12, 2024

Related U.S. Application Data
(60) Provisional application No. 63/514,100, filed on Jul. 17, 2023.

(51) Int. Cl. A01H 6/78

(52) U.S. Cl. CPC A01H 6/785
USPC PLT/202

(57) ABSTRACT

A new mandarin tree named ‘AC41114’ notable for its attractive, later-maturing seedless fruit.

Latin name: Citrus reticulata.

Common name: Mandarin.

Variety denomination: ‘AC41114’.

### BACKGROUND AND SUMMARY OF THE VARIETY

‘AC41114’ is a new mandarin tree that originated as an induced mutation of ‘Nadorcott’ mandarin (U.S. Plant Pat. No. 10,480) at Queensland, Australia in 2012. Bud sticks of ‘Nadorcott’ were irradiated in 2012, and subsequently grafted onto rootstock at Queensland, Australia in 2012.The new ‘AC41114’ mandarin tree was selected from among the grafted plants for further observation because of its attractive, later-maturing seedless fruit. The ‘AC41114’ mandarin tree has been observed to remain true to type over successive asexually propagated generations.

The ‘AC41114’ mandarin tree is distinguished from ‘W. Murcott’ by having no seeds and later maturity as compared to ‘W. Murcott.’

The ‘AC41114’ mandarin tree is distinguished from similar variety ‘Tango’ (U.S. Plant Pat. No. 17,863) as set forth in Table 1 below.

TABLE 1
Comparison of ‘AC41114’ Mandarin to ‘Tango’ Mandarin

| | ‘AC41114’ | ‘Tango’ |
|---|---|---|
| Growth habit | Spreading | Upright |
| Vigor | Medium to strong | Strong to very strong |
| Fruit rind color | Orange N25A | Orange 24A |
| Fruit glossiness | Strong to very strong | Strong |

### BRIEF DESCRIPTION OF THE PHOTOGRAPHS

The accompanying photographs, taken in 2023 and 2024 at Visalia, California, show an ‘AC41114’ mandarin tree grown on ‘Carrizo’ rootstock (not patented), and its fruit and flowers. The trees were planted in 2020. The colors of the claimed plant and its fruit may vary with lighting conditions. Therefore, color characteristics should be determined with reference to the observations described herein, rather than from these illustrations alone.

FIG. 1 shows sectioned fruit from an ‘AC41114’ mandarin tree;

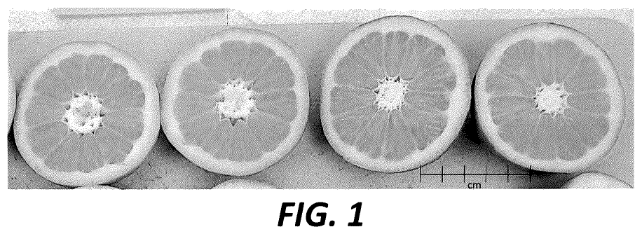

FIG. 2 shows a shoot from an ‘AC41114’ mandarin tree;

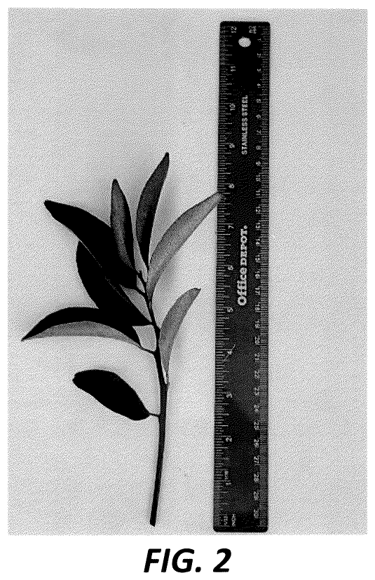

FIG. 3 shows leaves from an ‘AC41114’ mandarin tree;

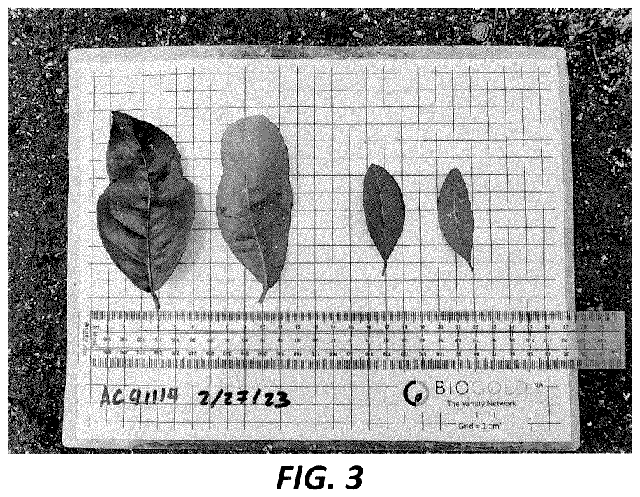

FIG. 4 shows the trunk of an ‘AC41114’ mandarin tree;

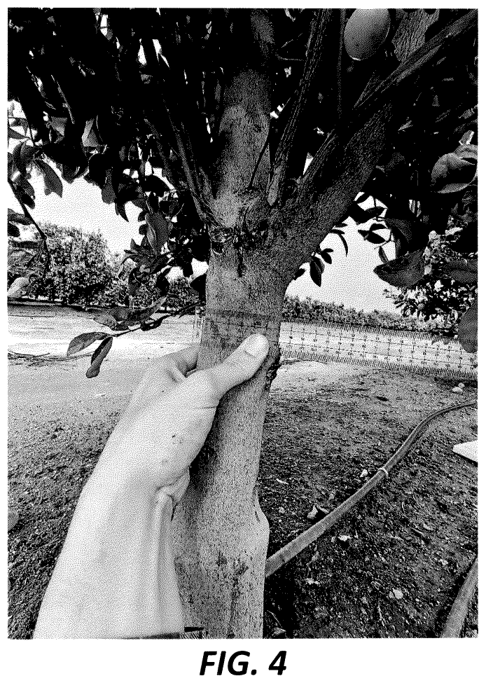

FIG. 5 shows an ‘AC41114’ mandarin tree; and,

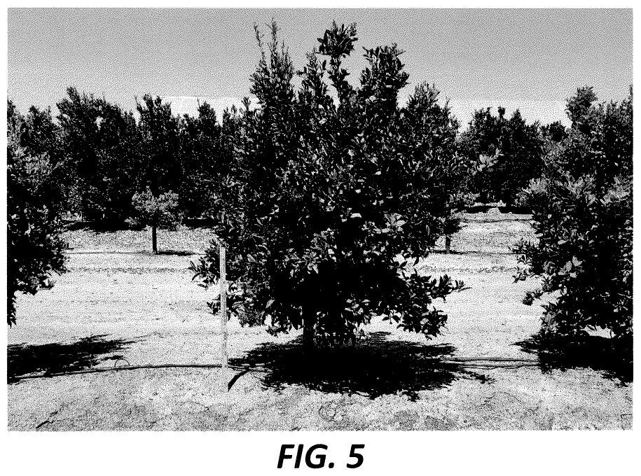

FIG. 6 shows flowers of an ‘AC41114’ mandarin tree.

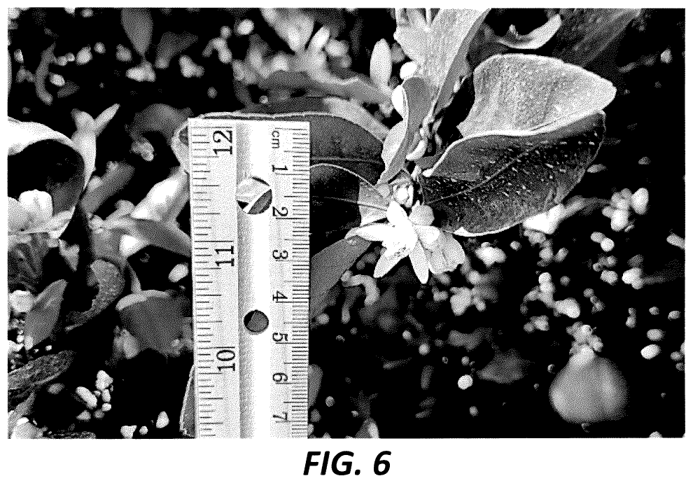

### DETAILED BOTANICAL DESCRIPTION

The following detailed botanical description is based on observations recorded at Visalia, California in 2023 and 2024 of ‘AC41114’ trees planted in 2020 on ‘Carrizo’ rootstock. The characteristics described will vary somewhat depending upon cultural practices, climatic conditions, location and growing season. Quantified measurements are expressed as an average of measurements taken from a number of individual plants of the claimed variety. The measurements of any individual plant or any group of plants of the variety may vary from the stated average. Colors are described with reference to The R.H.S. Colour Chart, 6th edition (Royal Horticultural Society 2019).

### DETAILED BOTANICAL DESCRIPTION

Tree:
   Vigor.—Medium strong.
   Density of canopy.—Medium dense.
   Habit.—Spreading.
   Height.—2 m to 3 m.
   Trunk diameter (at 30 cm above the graft).—5.5 cm to 6.5 cm.
   Bark texture.—Slightly rough.
   Bark color.—Grey-brown 199B.
Branch (fruiting branches about 1 m above graft):
   Length.—About 1 m.
   Diameter.—2 cm to 3 cm.
   Crotch angle.—Acute.
   Bark color.—Grey-brown 199B.
   Bark texture.—Slightly rough.
   Thorns.—Absent.
   Current year shoot length.—45 cm to 60 cm.
   Current year shoot color.—Green 137B.
Flower buds:
   Shape.—Oval.
   Length.—1.04 cm.
   Diameter.—0.58 cm.
   Color.—White 155C.
Flowers:
   Date of first bloom.—Apr. 10, 2023.
   Date of full bloom.—Apr. 17, 2023.
   Date of first fruitlet fall.—May 19, 2023.
   Number of flowers per cluster.—1 to 4.
   Number of petals per flower.—5.
   Flower diameter.—2.3 cm.
   Flower depth.—0.9 cm.
Petals:
   Shape.—Strap.
   Apex.—Acute.
   Margin.—Entire.
   Length.—1.1 cm.
   Width.—0.38 cm.
   Color—upper surface.—Green-white 157B.
   Color—lower surface.—White 155C.
   Relative position of margins.—Not overlapping.
Pistil:
   Quantity per flower.—1.
   Length.—9 mm.
Stigma:
   Length.—1.4 mm.
   Color.—Yellow 5A.
Style:
   Length.—5.8 mm.
   Color.—Yellow-green 145C.
Ovary:
   Length.—1.7 mm.
   Diameter.—2.8 mm.
   Color.—Yellow-green 144B.
Stamens:
   Quantity per flower.—15.
   Anther length.—1.0 mm to 1.5 mm.
   Anther color.—Yellow-orange 20B.
   Pollen.—Present.
Pedicel:
   Length.—4 mm.
   Pedicel diameter.—1 mm.
   Pedicel color.—Yellow-green 144B.
Sepals:
   Length.—2 mm.
   Width.—2.5 mm.
   Shape.—Deltoid with acuminate apex.
   Sepal margin.—Entire.
   Color—upper surface.—Yellow-green 144D.
   Color—lower surface.—Yellow-green 144C.
Leaves:
   Length.—65 mm to 80 mm.
   Width.—30 mm to 45 mm.
   Length/width ratio.—1.8 to 2.2.
   Blade margin.—Broadly crenate.
   Leaf shape.—Broadly elliptic.
   Apex shape.—Acute.
   Base shape.—Rounded.
   Texture of upper surface.—Medium-firm, smooth.
   Color of upper surface.—Yellow-green 147A.
   Color of lower surface.—Yellow-green 146A.
   Petiole length.—5 mm to 10 mm.
   Petiole diameter.—1 mm to 2 mm.
   Petiole color.—Yellow-green N144A.
   Wings.—Absent.
Fruit:
   Quantity per cluster.—2.
   Axial diameter.—76.3 mm.
   Apical diameter.—55.6 mm.
   General shape in profile.—Oblate.
   Shape in cross-section.—Round.
   Rind color.—Orange N25A.
   Rind thickness.—2.9 mm.
   Ease of peeling.—Easy.
   Rind texture.—Smooth to medium.
   Albedo color.—Yellow 15D.
   Quantity of fruit segments per fruit.—9 to 12.
   Toughness of segment membrane.—Weak.
   Juice sac length.—8 mm to 12 mm.
   Juice sac shape.—Elongated.
   Juice sac color.—Orange N25B.
   Juice soluble solids.—11.85° Brix.
   Harvest maturity.—January to May; after ‘Tango’ (U.S. Plant Pat. No. 17,863), a middle to late maturing variety.
   Seeds.—None.
   Market use.—Fresh.
   Keeping quality.—Excellent.
   Shipping quality.—Excellent.

We claim:

1. A new and distinct mandarin tree named ‘AC41114’ substantially as described and illustrated herein.
